SEQUENCE LISTING (6 sequences)

SEQ ID NO: 1 - DNA, 27 bp, artificial
  artificial primer sequence
ggcggccatatggtgagcaagggcgag

SEQ ID NO: 2 - DNA, 27 bp, artificial
  artificial primer sequence
ggcggccatatgtcccctatactaggt

SEQ ID NO: 3 - DNA, 30 bp, artificial
  artificial primer sequence
ggcggctgctcttccgcacttgtacagctc

SEQ ID NO: 4 - DNA, 30 bp, artificial
  artificial primer sequence
ggcggctgctcttccgcagtcacgatgcgg

SEQ ID NO: 5 - DNA, 26 bp, artificial
  artificial primer sequence
cggccgcatcgtgggtgctttgccaa

SEQ ID NO: 6 - DNA, 26 bp, artificial
  artificial primer sequence
ttggcaaagcacccacgatgcggccg

(19) United States
(12) Patent Application Publication
(10) Pub. No.: US 2004/0265921 A1
(43) Pub. Date: Dec. 30, 2004

(54) Intein-mediated attachment of ligands to proteins for immobilization onto a support

(75) Inventors: Marie-Laure Lesaicherre, Singapore (SG); Yee Peng Rina Lue, Singapore (SG); Mahesh Uttamchandani, Singapore (SG); Y.J. Grace Chen, Singapore (SG); Shao Qin Yao, Singapore (SG)

Correspondence Address: KLARQUIST SPARKMAN CAMPBELL, LEIGH & WHINSTON, LLP, One World Trade Center, 121 S.W. Salmon Street, Suite 1600, Portland, OR, 97204, US

(73) Assignee: National University of Singapore

(21) Appl. No.: 10/611,593

(22) Filed: Jun. 30, 2003

(51) Int. Cl. G01N 33/53

(52) U.S. Cl. 435/7.5

(57) ABSTRACT

The present invention provides an intein-mediated method of attaching a ligand to a protein for immobilization onto a support functionalized with an affinity receptor. In one embodiment, the ligand is biotin and the affinity receptor is avidin. Biotin is attached to the protein by reacting cysteine-biotin with a fusion protein comprising a cleavable intein and a protein of interest to effect cleavage of the cystein and attachment of biotin to the protein. The present invention further provides a novel protein array and a high throughput method of preparing protein arrays by expressing the protein of interest as an intein fusion protein including a binding domain for purification, attaching a ligand to the fusion protein under condition suitable for cleavage of the intein and attachment of the ligand to the remaining protein and immobilizing the resulting protein-ligand onto a support that has been functionalized with an affinity receptor.

### FIELD OF INVENTION

The present invention relates to protein arrays and to a method of preparing protein arrays. More particularly, the invention provides an intein-mediated method of immobilizing protein onto a support.

### BACKGROUND OF INVENTION

DNA microarray techniques are currently the method of choice for high-throughput analysis of nucleic acids. These techniques involve the screening of a partial or whole complement of an organism's transcribed genetic sequences by fixing gene sequences to be used as a probe in a discrete spot within a two-dimensional array of DNA spots. These microarrays can then be used to probe the mRNA expression profiles of particular biological samples.

However, the mRNA expression level in a cell does not always correlate well with the expressed protein levels. As well, mRNA levels do not give information pertaining to protein-protein or protein-ligand interactions or post-translational modifications of proteins. Therefore, the field of proteomics, the study of a cellular protein complement, is a rapidly emerging field. This field focuses on protein interactions and how proteins and their metabolites effect and influence different cellular events.

One of the major areas in proteomics is the development of high-throughput screening techniques for the discovery of novel protein functions, protein-protein interactions and protein-ligand interactions, and the identification of small molecules that disrupt these functions or mimic these interactions.

Currently, two-dimensional gel electrophoresis is widely used for high-throughput studies of proteins. However, this method is labor-intensive, technically challenging, and the obtained results can be difficult to reproduce. In addition, the dynamic range of this technique is limited and the method is ill-suited for membrane proteins. Out of the approximately 6000 yeast proteins, only about 3000 of them can be detected with two-dimensional gel electrophoresis.

The yeast two-hybrid system is also commonly used for high-throughput studies of protein-protein interactions. However, it is also a labor-intensive technique. In addition, it is not an accurate screening method, as it often gives false positive and false negative results.

The strategies used in the preparation of DNA microarrays are not directly applicable to peptide and protein microarrays. Using currently available amplification and purification techniques, it is possible to generate hundreds of DNA samples at one time for inclusion in a microarray. No strategy is currently available for the amplification of small molecules, peptides and proteins. Unlike DNA, small molecules, peptides and proteins do not bind to the surface of chips easily, and the binding chemistries used for DNA are not directly applicable to other types of molecules. As well, DNA is a much more stable molecule, making it easier to handle. Proteins are much more sensitive and will denature more readily under much less stringent conditions.

Microarrays of small molecules, peptides, and proteins for high-throughput proteomics studies are typically obtained with the aid of a commercially available high-precision robotic microarray spotter. Depending upon the type of spotters used and the spotting pins chosen, different sizes and types of molecules can be spotted, with varying degrees of spot density, onto a number of different solid surfaces, including glass slides, acrylamide gels and membranes.

CombiMatrix Corp (Seattle, USA) has developed peptide microarrays wherein the peptides are synthesized directly onto a solid glass chip, one amino acid at a time, using combinatory and semiconductor technology. This technique allows for very high-density arrays, up to one million sites per square centimetre. This method is similar to the one used by Affymetrix to generate their high-density DNA microarrays, or Genechips™. Due to the many intrinsic difficulties associated with peptide synthesis on a glass surface, the peptides that are directly synthesized on the chips are of poor quality. Consequently, peptide arrays of this type have yet to be adopted as a common screening technique in the field of proteomics.

Most small molecule and peptide based microarrays reported to date use non-specific, covalent immobilization of the molecules to the slide. While these methods may be applicable for such types of molecules, proteins need to be arrayed in a specific, ordered orientation in order to retain their full biological activity and ensure accessibility of their active sites with interacting molecules in the screening samples. As well, the conditions used to covalently attach molecules to the solid support tend to be quite harsh, and therefore unsuitable for the attachment of folded, active proteins to a solid surface.

Falsey (Bioconjugate Chem., 12:346-353 (2001)) discloses glyoxylyl-derivatized glass slides that are reacted with an oxime group in the small molecule or peptide so as to covalently attach the desired molecule to the glass slide. The conditions used for this reaction are fairly harsh, and have not been extended to full-length proteins.

Protein arrays have been prepared by random binding of proteins onto functionalized slides. As the orientation of each protein molecule is random, the proteins are not aligned so as to optimize interactions with target molecules. This method of attachment may also result in loss of or reduction in protein activity.

One report of site-specific attachment of proteins on glass slides (Zhu et al, Science, 293:2101-2105 (2001)) involves expressing each protein that is to be included in the micro-array with a GST domain and a 6-histidine tag fused to the N-terminus. The purified fusion proteins are then individually spotted onto Ni-NTA functionalized slides. Greater than 80% of the 3000 yeast proteins that were spotted were found to retain their full biological activities, presumably as a result of site-specific immobilization which ensures most proteins on the slide are oriented correctly. However, the binding between Ni-NTA and his-tag fusion proteins is neither very strong, nor very stable and susceptible to interference by many commonly used chemicals, making this immobilization method incompatible with many protein screening assays. Furthermore, the large GST domain fused to the N-terminus may interfere with folding of some proteins, as well as certain protein-protein interactions.

### SUMMARY OF INVENTION

In one aspect, the invention provides a method of immobilizing a protein onto a support comprising attaching a ligand to a fusion protein comprising a cleavable intein under condition suitable for the cleavage of the intein and attachment of the ligand to the remaining protein to form a protein-ligand and immobilizing the protein-ligand onto a support that is functionalized with an affinity receptor.

In another aspect, the invention provides a method of preparing a protein array comprising the steps of expressing a protein as a fusion protein comprising a cleavable intein and a binding domain downstream to the intein, contacting the expressed fusion protein with a substrate to which the binding domain binds, attaching a ligand to the fusion protein under condition suitable for cleavage of the intein and attachment of the ligand to the remaining protein to form a protein-ligand and immobilizing the protein-ligand onto a support that is functionalized with an affinity receptor.

The invention also provides a protein array comprising protein immobilized onto a support functionalized with an affinity receptor wherein the protein is attached to a ligand at the C-terminus by a peptide bond.

### BRIEF DESCRIPTION OF THE FIGURES

FIG. 1 shows intein-mediated expression, purification, biotinylation and site-specific immobilization of biotinylated proteins onto avidin-functionalized slides according to one embodiment of the invention.

FIG. 2 shows the structure of cysteine biotin used for intein-mediated biotinylation of proteins according to one embodiment of the invention.

FIG. 3 shows intein-mediated purification and biotinylation of maltose binding protein (MBP). (a) SDS-PAGE acrylamide gel stained for protein, with the following samples loaded in the respective lanes: (1) protein marker, (2) uninduced cell extract, (3) induced cell extract, (4) flow-through from column loading, (5) flow-through from column wash, (6) proteins bound to chitin column before cleavage, (7) flow-through from quick flush of cleavage agent, (8-9) first two elution fractions after overnight incubation at 4° C. with cysteine biotin, (10) remaining proteins bound to chitin column after cleavage. (b) Western blotting of biotinylated MBP using streptavidin-HRP for detection.

FIG. 4 shows the site-specific immobilization of biotinylated, functionally active proteins onto avidin slides. (a) EGFP, MBP and GST were individually detected with Cy3-anti-EGFP (green), Cy5-anti-MBP (red) and FITC-anti-GST (blue), respectively; (b) specific detection of all three proteins with a mixture containing all three antibodies; (c) fluorescence from the native EGFP; and (d) specific binding between GST and its Cy3-labeled natural ligand, glutathione. No binding between glutathione and EGFP/MBP was observed (data not shown).

FIG. 5 shows the strong binding of biotinylated proteins onto avidin-functionalized slides. Biotinylated GST was arrayed on an avidin slide and treated with different washing conditions: (a) 1 M acetic acid solution pH 3.3, (b) 60° C. water, (c) 4 M GuHCl, all for 30 min and (d) control slide with no treatment. Slides were probed with FITC-anti-GST.

### DETAILED DESCRIPTION OF PREFERRED EMBODIMENTS

The present invention uses an intein-mediated strategy to immobilize a protein onto a support. In one embodiment, the invention provides a method of immobilizing a protein onto a support comprising attaching a ligand to a fusion protein comprising a cleavable intein under condition suitable for the cleavage of the intein and attachment of the ligand to the remaining protein to form a protein-ligand and immobilizing the protein-ligand onto a support that is functionalized with an affinity receptor.

The term protein, as used herein refers to a polymer of amino acids that are linked by peptide bonds, and includes peptides, which generally refers to relatively small amino acid polymers, for example containing about 30 or fewer residues, or about 20 or fewer residues or about 10 or fewer residues. Where appropriate, the term peptide is used to specifically describe such amino acid polymers and to distinguish from larger proteins. As used within, the term “amino acids” refers to the standard set of genetically encoded L-amino acids (alanine, cysteine, aspartic acid, glutamic acid, phenylalnaine, glycine, histidine, isoleucine, lysine, leucine, methionine, asparagine, proline, glutamine, arginine, serine, threonine, valine, tryptopan and tyrosine), and derivatives thereof. In the context of polypeptides or peptides created by semi-synthetic or chemical methods, the term “amino acid” also refers to all non-natural amino acids, as well as the D-isomers of the genetically encoded amino acids.

The ligand may be any ligand that interacts with, for example by binding to an affinity receptor so as to form a ligand-affinity receptor complex. For example, the ligand may be a small molecule, protein, peptide, lipid or polynucleotide. The affinity receptor similarly may be any molecule that the ligand interacts with. Any receptor-ligand pair therefore may be suitable and include biotin-avidin, FLAG-antibody, GST-GSH, MBP-amylose, his-tags-Ni-NTA. Biotin-avidin is particularly preferred due to the strength and stability of the biotin-avidin interaction. Moreover, one skilled in the art will appreciate that certain receptor-ligand pair may not be suitable, for example if the ligand can have the effect of interfering with the function or structure of the protein that is to be immobilized.

Inteins, described in U.S. Pat. Nos. 5,981,182 and 5,834,247, the contents of which are incorporated by reference, are protein sequences embedded within a precursor protein that are removed by protein splicing. These sequences can be used to develop fusion protein expression systems to express and purify desired proteins. One such expression system which is commercially available from New England Biolabs (NEB) uses an intein from the Saccharomyces cerevisiae VMA gene which is mutated so that it only undergoes the first step of protein splicing to form a thioester (IMPACT system, pTYB vectors). In this system, a protein of interest is expressed as an N-terminal fusion to a mutated intein that also contains a chitin-binding domain on its C-terminus. The fusion protein can then be isolated on a chitin column and the desired protein is released by addition of a thiol agent, which cleaves the thioester, leaving the intein fragment on the chitin column.

In one embodiment, an intein fusion protein is prepared by cloning a DNA sequence encoding the protein of interest into a pTYB expression vector and expressing the resulting fusion protein in an appropriate bacterial host.

The techniques of the fusion protein expression and purification are well known in the art, and described for example in Sambrook et al. in “Molecular Cloning: A Laboratory Manual”, 3^{rd} Edition, Cold Spring Harbor Laboratory Press, and other laboratory manuals. In one embodiment, the fusion protein may be expressed from a pTYB expression vector in E. coli grown at 37° C. in Luria Bertani medium, using ampicillin to selectively maintain the cells transformed with the vector. Protein expression is induced using isopropylthiogalactoside. The cells are harvested and lysed and the cell debris is removed by standard methods known in the art. The supernatant of the crude cell lysate containing the fusion protein of interest may be passed over a chitin column and the column may be washed as appropriate.

In one embodiment, the ligand is biotin and may be attached to the fusion protein and the protein-biotin purified in a single step as set out in Tolbert (J. Am. Chem. Soc., 122:5421-5428 (2000)), incorporated herein by reference. The biotin may be attached to the fusion protein by adding a cysteine-containing biotin to the chitin column and allowing it to react with the bound fusion protein by stopping the column flow. Once the reaction is completed, the biotinylated protein is eluted from the chitin column. The cleaved intein and the affinity domain portion of the fusion protein remains bound on the column. As is known in the art, the fusion protein may also be purified and biotinylated using the affinity chromatography beads in batch form.

Any biotin derivative with an N-terminal cysteine (cysteine-biotin) may be used to attach to the protein since the N-terminal cysteine will react with the intein thioester, cleaving the intein, and undergo a nucleophilic rearrangement to form a peptide bond with the protein. The reaction therefore results in the intein fragment bound to the chitin column, and boitin is attached to the protein to be immobilized onto a support at the C-terminus by a peptide bond. Cysteine-biotin may be prepared by known methods using commercially available reagents, such as Boc- or Fmoc-protected cysteine and biotinyl compounds, for example, biotinylethylenediamine, as starting materials.

The protein-biotin is immobilized onto a support that has been functionalized with an affinity receptor. In one embodiment of the invention, the protein-biotin is immobilized onto a support by contacting the protein-biotin with an avidin-functionalized support. Numerous materials are suitable for use as support, for example: glass, silica, silicon and quartz.

Avidin as the term is used herein broadly refers to any avidin that may be derived from different organisms and includes streptavidin and any avidin modified to increase specificity of binding to biotin. As streptavidin is known to have higher nonspecific binding characteristics, in one embodiment, streptavidin can be used to functionalize a support.

The support may be affinity receptor-functionalized by covalently or non-covalently binding the affinity receptor to the surface of the support. In one embodiment, the support is avidin-functionalized by covalently or non-covalently binding avidin onto the support using methods known in the art. In one embodiment, avidin is covalently bound to a glass surface by reacting the glass surface with glycidoxypropyl trimethoxysilane and then reacting the resulting epoxy glass with avidin. Additional alternatives may be used to functionalize slides with avidin. For example, biotin may be bound to the surface as a support for avidin, as described by Falsey. Another approach is to functionalize the support with hydroxysuccinimide prior to covalent attachment of avidin.

The in vivo and in vitro biotinylation of proteins have previously been reported, but with limited success due to low yields and non-specific nature of the biotinylation reaction. In the system developed by NextGen Sciences, proteins are expressed from a vector encoding a 15-amino acid long sequence called BioTag™, which is specifically biotinylated by the biotin ligase enzyme. However this method has several constraints. The in vivo expression results in overexpression of fusion proteins and inclusion bodies in bacterial cells and the in vitro expression results in the degradation of the fusion proteins due to high levels of proteases. According to the present invention, by using an intein-mediated expression system, it is possible to express, purify and site-specifically biotinylate proteins for subsequent site-specific immobilization onto an avidin-functionalized solid support.

Thus far, the only reported method for site-specific attachment of proteins in an array has been the immobilization of his-tag proteins on slides functionalized with Ni-NTA. However, the binding between his-tag proteins and Ni-NTA complex is not very strong, and incompatible with many common chemicals such as DTT, SDS, EDTA, etc. The binding is also depleted outside the 4 to 10 pH range, or when the buffer contains high concentrations of common salts. In contrast, the binding between biotin and avidin is one of the strongest known, with dissociation constant of approximately 10⁻¹⁵ M. This interaction is stable under most stringent conditions. Avidin itself is also extremely stable, making it an ideal agent for slide functionalization. In addition, the interaction between avidin and biotin is instantaneous, hence requiring no incubation for protein immobilization. Therefore, a large number of proteins can be immobilized onto a support by a highly robust and stable interaction with avidin.

An important issue in generating a protein array is to ensure that proteins maintain their native activity. Proteins which are immobilized onto a support according to the invention have been shown to retain their native activity. Accordingly, the method of the present invention is ideally suited for preparing a protein array. Furthermore, a large number of proteins may be prepared in a high-throughput manner for immobilization onto a support by expression, purification and biotinylation of intein-fusion protein as described above, further facilitating the preparation of a protein array.

In one embodiment, the invention provides a method of preparing a protein array comprising the steps of expressing a protein of interest as a fusion protein comprising a cleavable intein and a binding domain downstream of the intein, contacting the expressed fusion protein with a substrate to which the binding domain binds, attaching a ligand to the protein under condition suitable for cleavage of the intein and attachment of the ligand to the remaining protein to form a protein-ligand and immobilizing the protein-ligand onto an affinity receptor-functionalized support.

Suitable support materials in the preparation of a protein array will be apparent to those skilled in the art and include glass, silicon, silica, quartz, carbon, metals, such as gold, platinum, aluminum, copper, titanium and their alloys. The binding domain, as described above, may be a chitin binding domain and the substrate chitin column may be used purify the fusion protein. It will also be appreciated that the intein expression vector may be modified to include other suitable binding domains that will be apparent to one skilled in the art. Furthermore, it will be understood that other expression vectors which comprises an intein that has been mutated to only undergo the first step of protein splicing and which is spliced upon addition of an appropriate reagent may be used and reference to cleavable intein is intended to broadly refer to any such mutated intein.

The protein-ligand may be immobilized onto an affinity receptor-functionalized support by spotting onto the support using conventional arraying techniques and equipment. A two-dimensional array is preferred as this arrangement allows for a greater number of proteins to be screened at a single time, and optimizes the spot to surface area ratio on the solid support. Within the array, each spot may contain a different protein of interest, or different spots may contain the same protein of interest, as is required for any particular array. The array may contain proteins of interest that comprise an entire or a partial proteome of a particular cell or organism.

The protein arrays of this invention may be used to screen for interactions between the immobilized proteins of interest and one or more protein targets. Protein targets may include proteins (including antibodies, enzymes and receptors), drugs, small molecules, hormones, biological molecules (including lipids) and other specific protein ligands.

In one embodiment, the invention provides a protein array comprising protein immobilized onto a support functionalized with an affinity receptor wherein the protein is attached to a ligand at the C-terminus by a peptide bond. In one embodiment the ligand is biotin and the affinity receptor is avidin, for example streptavidin, and the support may be glass.

Although the pTYB system provides a convenient system for the intein-mediated attachment of a ligand to a protein that is to be arrayed onto a solid support, any cloning system that contains a cloning site, a mutated intein sequence that undergoes only the first step of intein cleavage and a C-terminal binding domain is also suitable.

Moreover, while intein-mediated attachment of biotin to protein has been described, any ligand may be similarity treated to be attached to an intein-fusion protein to form a protein-ligand that can be immobilized onto a support functionalized with an affinity receptor. For example, any ligand covalently linked to the carboxylate group of the cysteine may be attached to the protein, at the same time effecting intein-medicated cleavage of the fusion protein. Any ligand containing a free thiol group that forms a thioester bond with the protein and an amino group which can undergo rearrangement to form an amide bond may also be used.

All documents referred to herein are fully incorporated by reference.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. All technical and scientific terms used herein have the same meaning as commonly understood by one of ordinary skill in the art of this invention, unless defined otherwise.

The word “comprising” is used as an open-ended term, substantially equivalent to the phrase “including, but not limited to”. The following examples are illustrative of various aspects of the invention, and do not limit the broad aspects of the invention as disclosed herein.

### EXAMPLES

### Example 1

### Functionalization of Glass Slide with Avidin

First, glass slides were cleaned in a piranha solution and derivatized with a 1% solution of (3-glycidoxypropyl) trimethoxisilane (95% ethanol, 16 mM acetic acid) for 1 hr and cured at 150° C. for 2 hours. The resulting epoxy slides were reacted with a solution of 1 mg/mL avidin in 10 mM NaHCO₃ for 30 minutes, washed with water, air dried and the remaining epoxide is quenched with a solution of 2 mM aspartic acid in a 0.5 M NaHCO₃ buffer (pH 9).

### Example 2

### Chemical Synthesis of Cysteine-biotin

Cysteine-biotin (FIG. 2) was synthesized with either (1) Boc-protected cysteine, or (2). Fmoc-protected cysteine.

(1) N-α-t-Boc-S-trityl-L-cysteine (1.2 g, 2.6 mmol), TBTU (1.0 g, 3.10 mmol), and HOBt (0.60 g, 3.9 mmol) were dissolved in 50 mL of dry DMF. This mixture was stirred under argon for 20 min at room temperature before addition of 4-methyl morpholine (0.75 g, 7.8 mmol) and biotinylethylenediamine (0.75 g, 2.6 mmol). The reaction was further stirred for 3 h, followed by evaporation in vacuo. The crude product was dissolved in 200 mL of CH₂Cl₂, extracted with 3×200 mL of H₂O, dried over MgSO₄, and concentrated in vacuo. Further purification was done by flash chromatography (4-8% MeOH in CH₂Cl₂, to give the protected form of cysteine-biotin, which was deprotected by first stirring in a solution containing trifluoroacetic acid (50 mL), H₂O (1.6 mL), and tri-isopropyl silane (1.2 g, 7.8 mmol) for 30 min, and then evaporated in vacuo. The resulting residue was taken in a mixture of 1:1H₂O/CH₂Cl₂ (200 mL), and the aqueous layer was extracted with 3×100 mL of CH₂Cl₂ before evaporating to dryness. N-α-t-Boc-S-trityl-L-cysteine, TBTU, HOBt, DMF, 4-methyl morpholine, biotinylethylenediamine, CH₂Cl₂, MgSO₄, MeOH, trifluoroacetic acid, tri-isopropyl silane, are all commercially available from chemical suppliers such as Sigma-Aldrich, Fisher Scientific, Merck, etc.

(2) N-Fmoc-S-Trityl-L-cysteine (0.996 g, 1.7 mmol), TBTU (0.674 g, 2.1 mmol) and HOBt (0.3989 g, 2.6 mmol) were dissolved in 17 mL of DMF. After stirring for 30 minutes at room temperature, biotinylethylenediamine (0.5 g, 1.7 mmol) and triethylamine (0.515 g, 5.1 mmol) were added. The reaction was carried out under nitrogen for 3 hours at room temperature, followed by concentration in vacuo. The resulting residue was dissolved in ethyl acetate (50 mL), and extracted with 1.0 M HCl (50 mL), 10% Na₂CO₃ (50 mL), saturated NaCl (50 mL), dried over MgSO₄, and then evaporated to dryness. A solution of 20% piperidine in DMF (15 mL) was added to the resulting residue and stirred for 30 minutes at room temperature. Following evaporation, the residue was dissolved in ethyl acetate and washed with 2×10% Na₂CO₃ (50 mL), saturated NaCl (50 mL), dried over MgSO₄, and then evaporated to dryness. The residue was taken in 15 mL of TFA/EDT/H₂O (9/0.5/0.5), stirred for 1 hour, and then evaporated to dryness. The residue was taken in 100 mL of 1:1 DCM/H₂O and insoluble solid was removed by filtration. N-Fmoc-S-Trityl-L-cysteine, TBTU, HOBt, DMF, biotinylethylenediamine, triethylamine, ethyl acetate, HCl, Na₂CO₃, NaCl, MgSO₄, piperidine, ethyl acetate, DCM are all commercially available from chemical suppliers such as Sigma-Aldrich, Fisher Scientific, Merck, etc.

Final purification of the product from both syntheses was done using HPLC with a C18 reverse-phase column to give the final product as a white solid (69% & 39% overall yield for Boc- & Fmoc-synthesis, respectively). ¹H NMR (400 MHz, D₂O) δ4.57 (dd, 1H, J=7.8, 5.0), 4.39 (dd, 1H, J=7.8, 5.0), 4.12 (t, 1H, J=5.4), 3.45 (m, 1H), 3.33-3.24 (m, 4H), 3.03 (dd, 1H, J=14.9, 5.4), 3.00-2.93 (m, 2H), 2.74 (d, 1H, J=13.2), 2.22 (t, 2H, J=7.3), 1.72-1.50 (m, 4H), 1.48-1.31 (m, 2H); ¹³C. NMR δ179.62, 170.46, 64.53, 62.70, 57.79, 56.01, 42.16, 42.12, 41.45, 37.96, 30.39, 30.12, 27.50, 27.30; ESI 390.2 (MH⁺).

### Example 3

### Cloning of Target Genes into pTYB1 Expression Vector

To construct EGFP-intein and GST-intein fusion proteins, EGFP and GST gene fragments were first PCR amplified from pEGFP (CLONTECH, USA) and pGEX-4T1 (Pharmacia Biotech, USA), respectively, and cloned into the expression vector pTYB1 (NEB, USA). PCR amplification for both EGFP and GST gene fragments utilized upstream primers (5′-GGC GGC CAT ATG GTG AGC AAG GGC GAG-3′) [Seq ID No. 1] & (5′-GGC GGC CAT ATG TCC CCT ATA CTA GGT-3′) [Seq ID No. 2] containing an Nde I site with a translation initiation codon (ATG), and downstream primers (5′-GGC GGC TGC TCT TCC GCA CTT GTA CAG CTC-3′) [Seq ID No. 3] & (5′-GGC GGC TGC TCT TCC GCA GTC ACG ATG CGG-3′) [Seq ID No. 4] containing a Sap I site, respectively. PCR mixtures (100 μI) contained 10 μl of 10x Deep Vent DNA polymerase buffer (NEB, USA), 4 mM magnesium sulfate, 10 mM of each dNTPs (Promega), 1 μM of each primer, 100 ng of plasmid DNA template and 5 units of Deep Vent DNA polymerase (NEB, USA). Amplification was carried out with a DNA Engine™ thermal cycler (MJ Research, USA) at 94° C. for 45 sec, 65° C. for 45 sec and 72° C. for 1 min, for 25 cycles. The PCR products were double digested with Nde I and Sap I (NEB, USA) and cloned into pTYB1 vector, via Nde I and Sap I sites, to yield the EGFP-intein and GST-intein constructs. The C-terminal residue of GST in pTYB1-GST-intein was site-mutagenized from Cys to Gly using QuikChange XL Site-Directed Mutagnesis Kit (Stratagene, USA) with upstream primer (5′-CGG CCG CAT CGT GGG TGC TTT GCC AA-3′) [Seq ID No. 5] and downstream primer (5′-TT GGC AAA GCA CCC ACG ATG CGG CCG-3′) [Seq ID No. 6]; Gly is underlined in the primers. The pTYB1 containing MBP-intein fusion protein, pMYB5, is commercially available (NEB, USA). The resulting T7-driven expression plasmids, pTYB-EGFP-intein, pTYB-GST-intein, and pTYB-MBP-intein, were then transformed into E. coli ER2566 host (NEB, USA) for protein expression.

### Example 4

### Expression of Fusion Proteins

Three proteins (MBP, EGFP and GST) were expressed as fusion proteins with the intein affinity tag at their C-termini. The transformed E. coli host was grown in Luria Bertani (LB) medium supplemented with 100 μg/ml of ampicillin at 37° C. in a 250 rpm shaker to an OD₆₀₀ of ˜0.6. Protein expression was induced for overnight at room temperature using 0.5 mM isopropyl thiogalactosidase (IPTG). Cells were harvested by centrifugation (5000×g, 15 min 4° C.), resuspended in lysis buffer (20 mM Tris-HCl pH 8.0, 0.5 M NaCl and 1 mM EDTA) and lysed by sonication (ultrasonic liquid processor model XL 2020) on ice. The cell debris was pelleted down by centrifugation (20,000×g, 30 min, 4° C.) to give a clear lysate ready for loading onto a column packed with chitin affinity resin (NEB, USA) for purification and biotinylation.

### Example 5

### Affinity Purification & C-Terminal Biotinylation of Expressed Proteins

Various chemical ligations were developed in the early 90s in order to help in the synthesis of long non-protected peptides. Uniquely reactive functionalities can be incorporated into each peptide by chemical synthesis to allow for the site-specific reactions of unprotected peptides. This chemical ligation has proven to be easy to implement and a variety of ligation chemistries have been used. However, this resulted in the incorporation of unnatural groups such as oxime, thiazolidine ring or thioester at the site of ligation between two peptide segments.

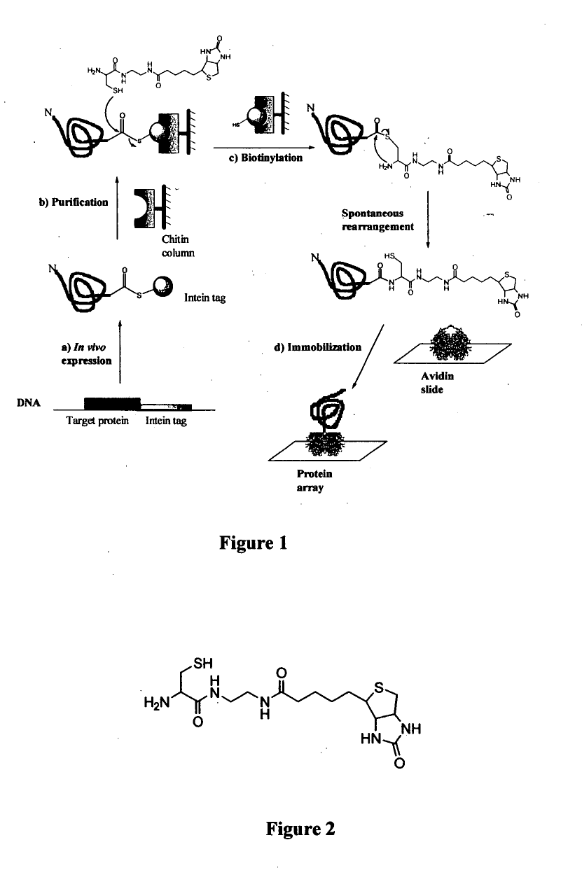

The native chemical ligation, developed by Stephen Kent's group (Dawson et al., Science 266: 776-779 (1994)), takes place between the N-terminal cysteine of one peptide and the thioester of a second peptide, eventually resulting in the formation of a stable peptide bond (FIG. 1). A feature of the native chemical ligation is that ligation occurs at a unique N-terminal cysteine, even if the two peptides contain other cysteine residues. Uniquely, the thioester-linked intermediate involving the N-terminal cysteine residue is able to undergo nucleophilic rearrangement by a highly favorable intramolecular mechanism; this step is irreversible and gives a polypeptide product, which is linked by a native peptide bond.

The purification protocol outlined in the IMPACT manual (NEB, USA) was followed with minor modifications. All purification procedures were carried out at 4° C. The column, packed with 3 ml of chitin beads, was pre-equilibrated with 30 ml of column buffer (20 mM Tris-HCl pH 8.0, 500 mM NaCl, 1 mM EDTA). The cleared cell lysate was loaded onto the column at a flow rate of 0.5 ml/min and washed with 50 ml of column buffer at a flow rate of 2 ml/min. For protein biotinylation, 6 mL of the column buffer containing 30 mM of the cysteine-containing biotin, was quickly flushed through the column to distribute it evenly throughout the resin before the flow was stopped and the column was incubated at 4° C. overnight. The resulting biotinylated protein was eluted with 10 ml of column buffer and collected in 1 ml fractions. The first fraction was discarded as it usually contains some cysteine-biotin. The 2^{nd} and 3^{rd} fractions were pooled and used for subsequent spotting on the array without any further treatment. Trace amount of cysteine-biotin in these fractions did not seem to affect the spotting quality, as NAP-5 treated protein samples from these fractions did not noticeably improve the array quality. The protein concentration of each fraction was determined by Bio-Rad protein assay (Bio-Rad, USA) and the purity of the column products were analyzed with 10% SDS-PAGE gel. Silver staining of the gel was done to visualize the separated protein bands. Biotinylation of the proteins were unambiguously confirmed by western blotting with streptavidin-conjugated HRP. The SDS-PAGE gel was electroblotted onto a polyvinylidene difluoride (PVDF) membrane (BiORad, USA) before blocking with 5% non-fat dry milk in PBST (0.1% Tween 20 in phosphate buffered saline, pH 7.4). The membrane was incubated with horseradish peroxidase (HRP)-conjugated streptavidin before visualized with an Enhanced ChemiLuminescent (ECL) kit (Amersham Pharmacia Biotech, USA).

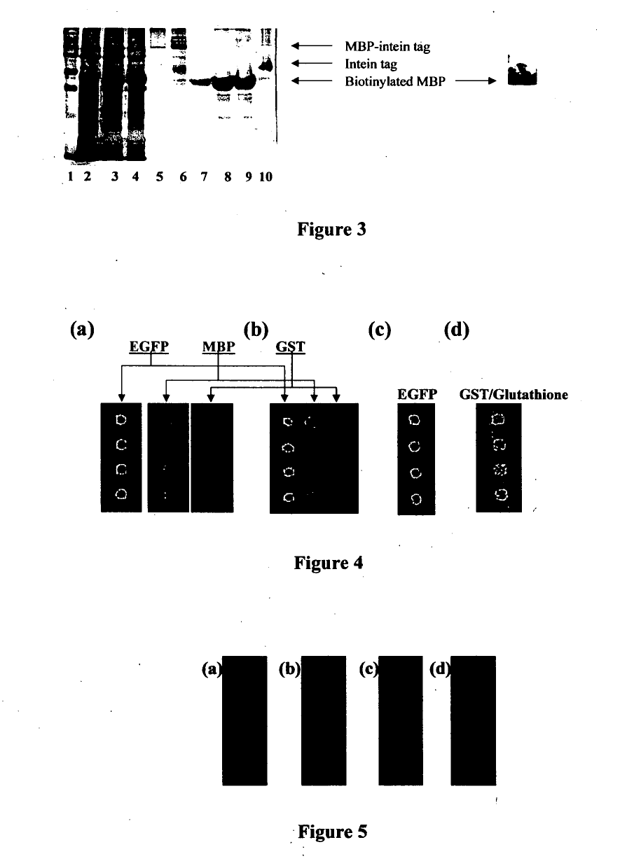

Based on SDS-PAGE (FIG. 3), the biotinylation reaction took place with 90-95% efficiency, generating proteins in sufficient purity (>95%) which were spotted directly, without any further treatment, onto an avidin-functionalized slide to obtain the corresponding protein array.

### Example 6

### In Vitro Expression

By expressing proteins in vitro, it is possible to rapidly, and in a very high throughput fashion, express and site-specifically biotinylate proteins to generate in a very short time high density arrays of site specifically arrayed proteins.

### Example 7

### Site-Specific Immobilization of Intein-Mediated Biotinylated Proteins

The inventors took advantage of the interaction between avidin and biotin, one of the strongest known non-covalent interactions (K_{d=}10⁻¹⁵M) to immobilize N-terminally biotinylated peptides and biotinylated proteins onto a glass slide functionalized with avidin. Avidin is also a highly stable protein that maintains its functions even under extremely harsh conditions, and therefore is an ideal candidate for slide derivatization.

Three proteins (MBP, EGFP and GST) were expressed in vivo as fusion proteins with an intein tag (intein fused to chitin binding domain) at their C-termini. The proteins were purified and biotinylated, in one single step, by first loading the crude cell lysate onto a column packed with chitin beads, then flushing the column with biotinylated cysteine to obtain the C-terminally biotinylated proteins. The proteins were subsequently spotted directly, without any further treatment, onto an avidin-functionalized slide to obtain the corresponding protein array.

A protein array was generated with the biotinylated EGFP, MBP and GST, and probed with Cy3-anti-EGFP, Cy5-anti-MBP and FITC-anti-GST, respectively. Three corresponding non-biotinylated proteins were also spotted onto the same slide, as controls, and the array was incubated with either individual antibodies (FIG. 4, a), or a mixture of all three antibodies (FIG. 4, b). Only specific binding between the biotinylated proteins and their corresponding antibodies were observed, regardless of the presence of other proteins (FIG. 4, a) and antibodies (FIG. 4, b), indicating the specific immobilization and versatility of this new protein array. Furthermore, no fluorescence signal was observed with the non-biotinylated control proteins (data not shown), confirming the essence of biotinylation for protein immobilization.

### Example 8

### Testing the Stability of Biotinylated Protein Microarray

In order to confirm the benefit of the avidin-biotin linkage, slides immobilized with GST were first subjected to a number of harsh washing conditions, and then incubated with FITC-labeled anti-GST to detect for any loss of GST on the surface. No loss of GST was observed even after the slide had been treated with (1) 1M acetic acid at pH 3.3, (2) 60° C. water and (3) 4 M GuHCl for prolonged time (FIG. 5) in sharp contrast with that of his-tag proteins on a Ni-NTA slide. For comparison, we have also prepared Ni-NTA slides according to published protocols. Briefly, epoxy slides were incubated with NTA dissolved in NaHCO₃. The slides were washed in water and soaked in 100 mM NiSO₄ for at least 1 hour, washed with 0.2 M acetic acid, 100 mM NaCl to give the Ni-NTA slides. We expressed a GFP fusion protein with a his-tag, and spotted it onto Ni-NTA slides as described. When this GFP-containing slide was treated with any of the above harsh conditions, the immobilization of the his-tag protein on the Ni-NTA was completely removed. More recent experiments have indicated that the his-tag/Ni-NTA immobilization does not even sustain simple aqueous washings.

What is claimed is:

1. A method of immobilizing a protein onto a support comprising:
(i) attaching a ligand to a fusion protein comprising a cleavable intein under condition suitable for the cleavage of the intein and attachment of the ligand to the remaining protein to form a protein-ligand; and
(ii) immobilizing the protein-ligand onto a support that is functionalized with an affinity receptor.

2. The method according to claim 1 wherein the ligand is biotin and the affinity receptor is avidin.

3. The method according to claim 2 wherein the support is glass.

4. The method according to claim 3 wherein the fusion protein is expressed from the expression vector pTYB 1.

5. The method according to claim 4 wherein the step of attaching the ligand comprises reacting the fusion protein with cysteine-biotin.

6. The method according to claim 5 wherein the glass is functionalized with avidin by reacting the glass surface with an epoxy silane compound and reacting the resulting surface with avidin.

7. The method according to claim 6 wherein the epoxy silane compound is glycidoxypropyl trimethoxysilane.

8. The method according to claim 7 wherein avidin is streptavidin.

9. A method of preparing a protein array comprising the steps of
(i) expressing a protein as a fusion protein comprising a cleavable intein and a binding domain downstream to the intein,
(ii) contacting the expressed fusion protein with a substrate to which the binding domain binds,
(iii) attaching a ligand to the fusion protein under condition suitable for cleavage of the intein and attachment of the ligand to the remaining protein to form a protein-ligand,
(iv) immobilizing the protein-ligand onto a support that is functionalized with an affinity receptor.

10. The method according to claim 9 wherein the ligand is biotin and the affinity receptor is avidin.

11. The method according to claim 10 wherein the fusion protein is expressed from the expression vector pTYB 1.

12. The method according to claim 11 wherein the substrate is a chitin column.

13. The method according to claim 12 wherein the step of attaching the ligand comprises adding cysteine-biotin to the chitin column.

14. The method according to claim 13 wherein the support is glass.

15. The method according to claim 14 wherein the affinity receptor is streptavidin.

16. The method according to claim 15 wherein immobilizing the protein-ligand comprises spotting the protein-ligand onto the support.

17. A protein array comprising protein immobilized onto a support functionalized with an affinity receptor wherein the protein is attached to a ligand at the C-terminus by a peptide bond.

18. The protein array according to claim 17 wherein the ligand is biotin and the affinity receptor is avidin.

19. The array according to claim 18 wherein the support is glass.

20. The protein array according to claim 19 wherein avidin is streptavidin.
